(19) United States
(12) Patent Application Publication
(10) Pub. No.: US 2015/0296774 A1
(43) Pub. Date: Oct. 22, 2015

(54) ANTIMICROBIAL COMPOSITIONS

(71) Applicant: Takasago International Corporation, Tokyo (JP)

(72) Inventors: Raphael KL. Kang, Leonia, NJ (US); Alba T. Cilia, River Edge, NJ (US)

(73) Assignee: Takasago International Corporation, Tokyo (JP)

(21) Appl. No.: 14/753,704

(22) Filed: Jun. 29, 2015

Related U.S. Application Data
(62) Division of application No. 13/677,819, filed on Nov. 15, 2012.
Application No. 13/677,819 is a continuation of application No. PCT/US2011/037600, filed on May 23, 2011.
(60) Provisional application No. 61/347,439, filed on May 23, 2010.

(51) Int. Cl. A01N 35/02; A61L 9/01

(52) U.S. Cl. CPC A01N 35/02; A61L 9/01; A61L 2209/21

(57) ABSTRACT

Antimicrobial compositions that include at least one aliphatic aldehyde component and allyl isothiocyanate are provided. Methods of reducing bacterial activity using the instantly disclosed compositions are also provided.

### CROSS REFERENCE TO RELATED APPLICATIONS

This application claims priority to and is a divisional of U.S. patent application Ser. No. 13/677,819, filed Nov. 15, 2012, which is a continuation of International Patent Application No. PCT/US2011/037600 filed May 23, 2011, which claims priority to U.S. Provisional Application Ser. No. 61/347,439, filed May 23, 2010, the contents of which are hereby incorporated by reference in their entireties herein.

### FIELD

The disclosed subject matter relates to compositions having antimicrobial activity, and methods of reducing the antimicrobial activity of a solid, liquid or surrounding air space that employ such compositions.

### BACKGROUND

Mustard essential oil (MEO) has been described as a defense mechanism against herbivores in plants of the Crucifereae family. While the antimicrobial activity of MEO and its primary component allyl isothiocyanate (AIT), individually, has been reported in various studies, there remains a need for antimicrobial compositions with increased efficacy, and that reduce the amount of MEO required to achieve sufficient antimicrobial activity. There also remains a need for antimicrobial compositions that are active against gram-negative and gram-positive bacteria in the vapor phase.

### SUMMARY

It has been found that the combination of an aliphatic aldehyde and allyl isothiocyanate, the active component of mustard essential oil, provides increased antimicrobial activity against both gram-negative and gram-positive bacteria. Accordingly, one aspect of the presently disclosed subject matter provides an antimicrobial composition that includes at least one aliphatic aldehyde component and allyl isothiocyanate.

In one embodiment, aliphatic aldehyde component is a C₆-C₁₃ aldehyde, or a C₇-C₁₂ aldehyde, or a C₇-C₁₁ aldehyde, or a C₉-C₁₃ aldehyde, or a C₁₀-C₁₂ aldehyde. These aldehyde components can be unsaturated (e.g., α, β unsaturated aldehyde) and/or these aldehyde components can be straight-chained (i.e., unbranched). The allyl isothiocyanate can be obtained from mustard essential oil, it can be obtained from other sources, or added as a pure, or relatively pure component to the antimicrobial composition.

In one embodiment, the antimicrobial composition includes from about 5 wt % to about 40 wt %, or from about 8 wt % to about 12 wt %, of an unsaturated or saturated C₆-C₁₃ straight-chained or branched aliphatic aldehyde.

Another aspect of the presently disclosed subject matter provides a method of reducing the bacterial activity of an environment that includes applying any one (or more) of the antimicrobial composition of the present application. In one embodiment the antimicrobial composition is applied in the vapor phase (e.g., the composition is allowed to evaporate within a relatively confined space).

Another aspect of the presently disclosed subject matter provides a method of preserving a product against spoilage that includes applying any one (or more) of the antimicrobial composition of the present application. Another aspect of the presently disclosed subject matter provides a method of preventing malodor in a confined air space that includes introducing any one of the antimicrobial compositions disclosed herein to the confined air space.

Another aspect of the presently disclosed subject matter provides an air sanitizer comprising any one of the antimicrobial compositions disclosed herein. For example, the air sanitizer can be in a form capable of being maintained in close proximity to a toilet rim. The air sanitizer can be in the form of a polymeric bead, an oil or a gel. Yet another aspect of the presently disclosed subject matter provides packaging for foodstuff that includes any one of the antimicrobial compositions disclosed herein.

### BRIEF DESCRIPTION OF THE DRAWINGS

FIG. 1 depicts Minimum Inhibitory Concentrations (MIC) of trans-2-octenal and trans-2-octenal and mustard essential oil against gram-negative and gram-positive bacteria.

FIG. 2 depicts Minimum Inhibitory Concentrations (MIC) for C₆-C₁₁ trans-2-alkenals alone and in combination with mustard essential oil against gram-negative and gram-positive bacteria.

### DETAILED DESCRIPTION

### Aldehyde Component

The presently disclosed subject matter provides antimicrobial compositions that include at least one aldehyde component. In one embodiment, the aldehyde component is an aliphatic aldehyde.

In one embodiment, the aldehyde component is an aliphatic C₆-C₁₃ aldehyde, including unsaturated aliphatic C₆-C₁₃ aldehydes having 1, 2, 3, 4 or more double bonds (e.g., an aliphatic C₆-C₁₃ α, β unsaturated aldehyde). In one embodiment, the aldehyde component is an aliphatic C₇-C₁₂ aldehyde, including unsaturated aliphatic C₇-C₁₂ aldehydes having 1, 2, 3, 4 or more double bonds (e.g., an aliphatic C₇-C₁₂ α, β unsaturated aldehyde). In one embodiment, the aldehyde component is an aliphatic C₉-C₁₃ aldehyde, including unsaturated aliphatic C₉-C₁₃ aldehydes having 1, 2, 3, 4 or more double bonds (e.g., an aliphatic C₉-C₁₃ α, β unsaturated aldehyde). In one embodiment, the aldehyde component is an aliphatic C₇-C₁₁ aldehyde, including unsaturated aliphatic C₇-C₁₁ aldehydes having 1, 2, 3, 4 or more double bonds (e.g., an aliphatic C₇-C₁₁ α, β unsaturated aldehyde). In one embodiment, the aldehyde component is an aliphatic C₁₀-C₁₂ aldehyde, including unsaturated aliphatic C₁₀-C₁₂ aldehydes having 1, 2, 3, 4 or more double bonds (e.g., an aliphatic C₁₀-C₁₂ α, β unsaturated aldehyde).

In one embodiment, the aldehyde component is straight-chained, and not branched (e.g., a straight-chained unsaturated C₆-C₁₃ aldehyde, a straight-chained unsaturated C₇-C₁₂ aldehyde, a straight-chained unsaturated C₇-C₁₁ aldehyde, a straight-chained unsaturated C₉-C₁₃ aldehyde, a straight-chained unsaturated C₁₀-C₁₂ aldehyde, a straight-chained unsaturated C₁₀-C₁₂ aldehyde).

In an alternative embodiment, the aldehyde component is a branched unsaturated aliphatic aldehyde (e.g., a branched unsaturated C₆-C₁₃ aldehyde, a branched unsaturated C₇-C₁₂ aldehyde, a branched unsaturated C₇-C₁₁ aldehyde, a branched unsaturated C₉-C₁₃ aldehyde, a branched unsaturated C₁₀-C₁₂ aldehyde, or a branched unsaturated C₁₀-C₁₂ aldehyde). In one embodiment the alkyl chain of the branched aliphatic aldehyde is substituted with one or more of methyl, ethyl and/or propyl groups. An example of branched unsaturated aliphatic aldehyde applicable for use in the compositions of the present application include, 2,6-dimethyl-5-heptenal.

In a still alternative embodiment, the aldehyde component can be a saturated aldehyde, such as an unbranched (e.g., octanal, nonanal, decanal) or branched (e.g., 2-methyl undecanal) saturated C₆-C₁₃ aldehyde. Combinations of saturated aldehydes can be employed (e.g., a combination of octanal, nonanal and decanal aldehdyes), or they can be used alone with, or without unsaturated aldehydes (which themselves may be used alone, or in combination). In one embodiment, the aldehyde component is selected from one or more of: hexanal, heptanal, octanal, nonanal, decanal, undecanal, dodecanal, and tridecanal.

In one embodiment, the aldehyde component is a C₈ aldehyde (e.g., trans-2-octenal, 2, 4 octadienal). In an alternative embodiment, the aldehyde component is a C₁₂ aliphatic aldehyde (e.g., trans-2-dodecenal, 2, 4 dodecadienal, 2-methyl undecanal).

In one embodiment, hexenal, octanal, nonanal, and undecenal are excluded as aldehyde components.

In one embodiment, the aldehyde component is selected from:

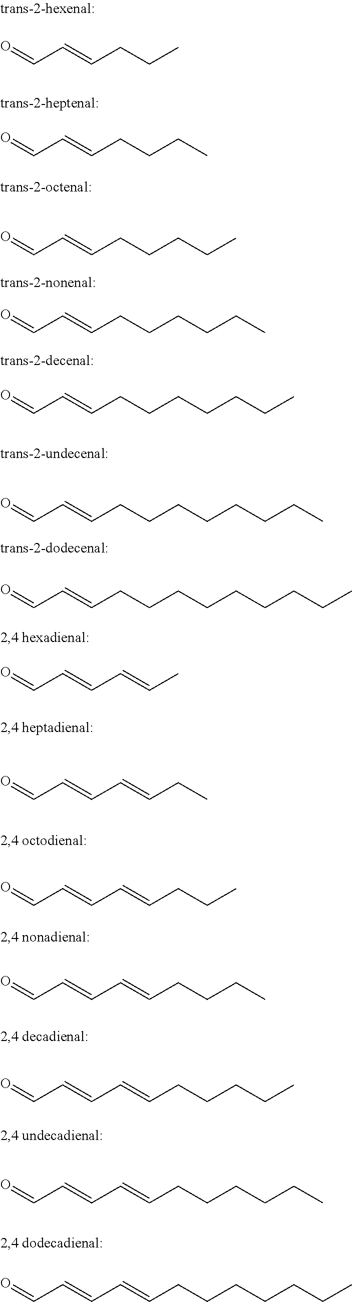

While the above-described aldehyde components can be used in combination with allyl isothiocyanate, in certain embodiments, the antimicrobial composition does not contain allyl isothiocyanate. For example, one embodiment of the presently disclosed subject matter provides a composition that includes from about 5 wt % to about 40% wt %, or from about 8 wt % to about 12 wt %, of one or more aldehyde components described herein (e.g., from about 5 wt % to about 40 wt %, or from about 8 wt % to about 12 wt %, of unsaturated or saturated C₆-C₁₃ straight-chained or branched aliphatic aldehyde).

### Allyl Isothiocyanate

In addition to an aldehyde component, the presently disclosed antimicrobial compositions can also include allyl isothiocyanate. Allyl isothiocyanate can be obtained, for example, from mustard essential oil, which in turn can be commercially obtained. While amounts will vary depending on the source, mustard essential oil typically contains greater than about 90 wt % of allyl isothiocyanate. Alternatively, allyl isothiocyanate can be added in pure, or relatively pure form to the composition.

Allyl isothiocyanate can also be obtained, for example, from brussels sprouts (about 0.1 mg/kg), cabbage (about 3 mg/kg), cauliflower (about 0.08 mg/kg), horseradish (about 1350 mg/kg) and mustard (about 400-15,000 mg/kg).

Allyl isothiocyanate can be obtained, for example, by pressing the seeds of brown mustard (Brassica juncea) to remove non-volatile oils. The residue of pressed seeds can be macerated with warm, deionized water and allowed to stand. The macerate can be distilled (e.g., via steam distillation) to yield a volatile fraction with >about 95 wt % allyl isothiocyanate.

### Antimicrobial Compositions

Other components suitable for use in an antimicrobial composition, and known to those of ordinary skill in the art, can be added to the antimicrobial compositions of the present application. The antimicrobial compositions of the present application are particularly active in the vapor phase. Thus, other antimicrobial components that are antimicrobially active in solution (i.e., the liquid phase), can be added to presently disclosed compositions to supplement the overall activity of the presently disclosed compositions. Furthermore, it has been found that α, β-unsaturated aldehydes, particularly C₉₋₁₃ α, β-unsaturated aldehydes, are active in solution and thus can be included to provide antimicrobial activity in solution (as well as the vapor phase).

The antimicrobial compositions of the present application can contain, for example, fragrance components, fillers, buffers, preservatives and other additives known to those of ordinary skill in the art. The aldehyde component and allyl isothiocyanate (for example allyl isothiocyanate obtained from mustard essential oil) can be diluted to use concentrations with an appropriate solvent. Since allyl isothiocyanate is decomposed by water, non-aqueous solvents are preferred (e.g., fragrance oils or glycols). Furthermore, the compositions should be stored and processed to avoid high heat, since excessive temperatures (e.g., above 170° C.) can also degrade the allyl isothiocyanate.

The compositions can be added to fragrance oils, flavor oils, and essential oils. In addition to liquids, the antimicrobial compositions of the present application can be incorporated within solids, such as plastics, paper and soap/detergent solid blocks (e.g., but not limited to, polymer beads, such as EVA beads, or gels, oils, etc.) according to techniques known to those of ordinary skill in the art.

Use amounts of the aldehyde component, allyl isothiocyanate and other components of the composition can be determined by persons of ordinary skill in the art. As non-limiting examples, the total amount of the aldehyde component(s) in the antimicrobial composition can range from about 2 wt % to about 80 wt %, or from about 5 wt %, to about 40 wt %. or from about 20 wt % to about 60 wt %, or from about 30 wt % to about 50 wt %, based on the total weight of the antimicrobial composition.

As non-limiting examples, the total amount of the allyl isothiocyanate in the antimicrobial composition can range from about 0.0001 wt % to about 40 wt %, or from about 0.1 wt % to about 5 wt %, or from about 0.05 wt % to about 0.5 wt %, based on the total weight of the antimicrobial composition.

Based on the efficacy of combining the aldehyde component with allyl isothiocyanate, the antimicrobial compositions can contain less allyl isothiocyanate (and when obtained from mustard oil, less mustard oil) than antimicrobial compositions of the prior art that contain allyl isothiocyanate, but do not contain an aldehyde component.

In embodiments which the allyl isothiocyanate is obtained from mustard essential oil, the weight ratio of the total amount of aldehyde component to mustard essential oil can range, for example, from about 0.1:1 to about 500:1, or from about 0.1:1 to about 100:1 (e.g., 50:1), or from about 0.5:1 to about 5:1 (e.g., about 1:1, or 3:1). Other ratios can be used depending on, among other things, the end use of the antimicrobial composition and the other components of the composition.

The antimicrobial compositions of the present application have been found to exhibit antimicrobial activity in the vapor phase against both gram-positive bacteria (e.g., Staphylococcus aureus, Enterococcus faecalis, Enterococcus hirae) and gram-negative bacteria (e.g., Escherichia coli, Salmonella enterica, Pseudomonas aeruginosa).

### Applications

The presently disclosed anti-microbial compositions can be employed in any application in which it is desired to reduce bacterial activity, such as, for example, as bathroom and kitchen cleaning and deodorizing products (e.g., as a dishwasher deodorizer for use in a dishwasher to reduce malodor). The presently disclosed compositions can be employed, for example, as a preservative for foodstuff, for malodor control (e.g., as an air sanitizer for a confined space), and as an antimicrobial agent. Because of the high vapor phase activity of the presently disclosed compositions, they are particularly suitable in enclosed spaces, such as bathroom applications (e.g., around the toilet), in locker rooms, closets and other confined spaces in which vapors emanating from the composition can be retained for a time sufficient to reduce bacterial activity.

Preferred applications include employing the compositions to control bacteria and/or malodor for use in small spaces/small rooms, and for use in dishwashers, refrigerator, garbage pails, sink garbage disposals, toilet bowls (e.g., as a toilet rim deodorizer), laundry hampers, diaper pails, closets, show boxes, (clothes/fabric) storage boxes, cat litter, pet litter boxes, pet cages, pet bedding, gym lockers, gym bags, sneakers, shoes, etc.

One embodiment of the presently disclosed subject matter provides a method of reducing the activity of Enterococcus hirae, comprising applying a composition that includes a C₉ to C₁₃ aldehyde component, such as any one of the C₉ to C₁₃ aldehyde components disclosed herein (or a combination thereof). Another embodiment of the presently disclosed subject matter provides a method of reducing the activity of Staphylococcus aureaus, comprising applying a composition that includes a C₁₀ to C₁₂ aldehyde component, such as any one of the C₁₀ to C₁₂ aldehyde components disclosed herein (or a combination thereof).

Based on the surprising benefits obtained from combining an aldehyde component with allyl isothiocyanate, one benefit of the presently disclosed compositions is that the amount of mustard essential oil required to provide active levels of allyl isothiocyanate can be reduced. Accordingly, one embodiment of the present application provides a method of increasing the antimicrobial activity of a composition containing allyl isothiocyanate (e.g., a composition containing mustard essential oil) that includes adding at least one, or a combination of aldehyde components to the composition, in which the aldehyde component can include any one of the presently described aldehyde components.

### EXAMPLES

The present invention is further described by means of the examples, presented below. The use of such examples is illustrative only and in no way limits the scope and meaning of the invention or of any exemplified term. Likewise, the invention is not limited to any particular preferred embodiments described herein. Indeed, many modifications and variations of the invention will be apparent to those skilled in the art upon reading this specification. The invention is therefore to be limited only by the terms of the appended claims along with the full scope of equivalents to which the claims are entitled.

### Example 1

Seeded brain heart infusion (BHI) agar plates of Escherichia coli ATCC 10536, Salmonella enterica ATCC 13311, Pseudomonas aeruginosa ATCC 15422, Staphylococcus aureus ATCC 6538, Enterococcus faecalis ATCC 29212, and Enterococcus hirae ATCC 10541 were placed in sealed 7 L acrylic boxes and exposed to vapors of trans-2-alkenals (C₆ to C₁₁ carbon chain length) and mustard essential oil (MEO) binary combinations.

The trans-2-alkenals and MEO were weighed neat into a glass jar which was positioned in the box between the seeded agar plates. The concentration of the single materials and binary combinations introduce in the 7 L box were expressed as weight per unit volume of the box (mg/L). Several concentrations ranging from 0.5 mg/L to 43 mg/L of the single materials and combinations were tested for each organism. The vapor phase minimum inhibition concentration (MIC) with “no growth” after 3 days of incubation at room temperature. Fractional Inhibition Concentrations (FIC) was determined to evaluate the antibacterial effect of trans-2-alkenals and MEO combinations in the vapor phase based on the following formula:

Combined FIC=(MIC of trans-2-alkenal_{combination}/MIC of trans-2-alkenalₐₗₒₙₑ)+(MIC of MEO_{combination}/MIC of MEOₐₗₒₙₑ)

MIC's of the trans-2-alkenal_{combination} was determined by starting with one-half or one-quarter of the MIC of the trans-2-alkenalₐₗₒₙₑ and one-half or one-quarter of the MIC of the MEOₐₗₒₙₑ, for the particular bacteria tested, and stepping down the concentration of the aldehyde until the minimum concentration is achieved such that the agar plate showed no bacterial growth (as observed by the naked eye). MIC's of the MEO_{combination} was determined by starting with one-half or one-quarter of the MIC of the trans-2-alkenalₐₗₒₙₑ and one-half or one-quarter of the MIC of the MEOₐₗₒₙₑ, and stepping down the concentration of the MEO until the minimum concentration is reached such that the agar plate showed no bacterial growth (as observed by the naked eye).

Combined FIC values for trans-2-alkenals and MEO combinations are shown below in Table 1. Combinations with combined FIC equal or less than 0.5 are considered to have a strong synergistic effect. Combinations with combined FIC equal to or less than 1, but greater than 0.5 are considered to exhibit synergistic properties.

TABLE 1
Combined FIC values for trans-2-
alkenals and MEO combinations (vapor phase)

| | trans-2- | trans-2- | trans-2- | trans-2- | trans-2- | trans-2- |
|---|---|---|---|---|---|---|
| | hexenal + | heptenal + | octenal + | nonenal + | decenal + | undecenal + |
| | MEO | MEQ | MEO | MEO | MEO | MEO |
| P.a. ATCC | 0.6 | 0.5 | 0.5 | 0.5 | 0.5 | 0.5 |
| 15422 | | | | | | |
| S.e. ATCC | 1.0 | 1.0 | 0.5 | 0.5 | 0.5 | 0.5 |
| 13311 | | | | | | |
| E.c. ATCC | 1.0 | 1.0 | 0.5 | 0.8 | 0.9 | 0.5 |
| 10536 | | | | | | |
| E.f. ATCC | 1.1 | 1.3 | 0.4 | 0.8 | 0.8 | 0.8 |
| 29212 | | | | | | |
| E.h. ATCC | 0.8 | 0.8 | 0.5 | 0.8 | 0.8 | 0.8 |
| 10541 | | | | | | |
| S.a. ATCC | 0.8 | 1.0 | 1.0 | 0.8 | 0.9 | 0.8 |
| 6538 | | | | | | |

MIC values used to obtain the FIC values in Table 1 is shown in Tables 2-5, below:

TABLE 2
Combined MIC and FIC values for trans-2-alkenals
and MEO combinations - E.h. 10541 (vapor phase)

| | | MIC trans- | | MIC MEO | |
|---|---|---|---|---|---|
| | MIC trans- | 2-alkenal in | | in | |
| | 2-alkenal | combination | MIC MEO | combina- | Com- |
| | alone | with MEO | alone | tion | bined |
| | (mg/L) | (mg/L) | (mg/L) | (mg/L) | FIC |
| trans-2- | 12.8 | 3.6 | 12.8 | 6.4 | 0.8 |
| hexenal + | | | | | |
| MEO | | | | | |
| trans-2- | 12.8 | 6.4 | 12.8 | 3.6 | 0.8 |
| heptenal + | | | | | |
| MEO | | | | | |
| trans-2- | 25.6 | 6.4 | 12.8 | 3.6 | 0.5 |
| octenal + | | | | | |
| MEO | | | | | |
| trans-2- | 6.4 | 3.6 | 12.8 | 3.6 | 0.8 |
| nonenal + | | | | | |
| MEO | | | | | |
| trans-2- | 6.4 | 3.6 | 12.8 | 3.6 | 0.8 |
| decenal + | | | | | |
| MEO | | | | | |
| trans-2- | 12.8 | 6.4 | 12.8 | 3.6 | 0.8 |
| undecenal + | | | | | |
| MEO | | | | | |

TABLE 3
Combined MIC and FIC values for trans-2-alkenals
and MEO combinations - E.f. 29212 (vapor phase)

| | | MIC trans- | | MIC MEO | |
|---|---|---|---|---|---|
| | MIC trans- | 2-alkenal in | | in | |
| | 2-alkenal | combination | MIC MEO | combina- | Com- |
| | alone | with MEO | alone | tion | bined |
| | (mg/L) | (mg/L) | (mg/L) | (mg/L) | FIC |
| trans-2- | 6.4 | 3.6 | 12.8 | 6.4 | 1.1 |
| hexenal + | | | | | |
| MEO | | | | | |
| trans-2- | 6.4 | 6.4 | 12.8 | 3.6 | 1.3 |
| heptenal + | | | | | |
| MEO | | | | | |
| trans-2- | 43.0 | 3.6 | 12.8 | 3.6 | 0.4 |
| octenal + | | | | | |
| MEO | | | | | |
| trans-2- | 6.4 | 3.6 | 12.8 | 3.6 | 0.8 |
| nonenal + | | | | | |
| MEO | | | | | |
| trans-2- | 6.4 | 3.6 | 12.8 | 3.6 | 0.8 |
| decenal + | | | | | |
| MEO | | | | | |
| trans-2- | 12.8 | 6.4 | 12.8 | 3.6 | 0.8 |
| undecenal + | | | | | |
| MEO | | | | | |

TABLE 4
Combined MIC and FIC values for trans-2-alkenals
and MEO combinations - P.a. 15442 (vapor phase)

| | | MIC trans- | | MIC MEO | |
|---|---|---|---|---|---|
| | MIC trans- | 2-alkenal in | | in | |
| | 2-alkenal | combination | MIC MEO | combina- | Com- |
| | alone | with MEO | alone | tion | bined |
| | (mg/L) | (mg/L) | (mg/L) | (mg/L) | FIC |
| trans-2- | 12.8 | 1.8 | 2.0 | 1.0 | 0.6 |
| hexenal + | | | | | |
| MEO | | | | | |
| trans-2- | 25.6 | 1.0 | 2.0 | 1.0 | 0.5 |
| heptenal + | | | | | |
| MEO | | | | | |
| trans-2- | 43.0 | 1.8 | 2.0 | 1.0 | 0.5 |
| octenal + | | | | | |
| MEO | | | | | |
| trans-2- | 43.0 | 0.9 | 2.0 | 3.0 | 0.5 |
| nonenal + | | | | | |
| MEO | | | | | |
| trans-2- | 43.0 | 1.8 | 2.0 | 1.0 | 0.5 |
| decenal + | | | | | |
| MEO | | | | | |
| trans-2- | 43.0 | 1.8 | 2.0 | 1.0 | 0.5 |
| undecenal + | | | | | |
| MEO | | | | | |

TABLE 5
Combined MIC and FIC values for trans-
2-octenal formulations (vapor phase)

| | | MIC trans- | | MIC MEO | |
|---|---|---|---|---|---|
| | MIC trans- | 2-octenal in | | in | Com- |
| | 2-octenal | combination | MIC MEO | combina- | bined |
| | alone | with MEO | alone | tion | FIC |
| P.a. 15442 | 43.0 | 1.8 | 2.0 | 1.0 | 0.5 |
| S.e. 13311 | 43.0 | 1.8 | 2.0 | 1.0 | 0.5 |
| E.c. 10536 | 43.0 | 1.8 | 2.0 | 1.0 | 0.5 |
| E.f. 29212 | 43.0 | 6.4 | 12.8 | 3.6 | 0.4 |
| E.h. 10541 | 25.6 | 6.4 | 12.8 | 3.6 | 0.5 |
| S.a. 6538 | 3.6 | 1.8 | 2.0 | 3.0 | 1.0 |

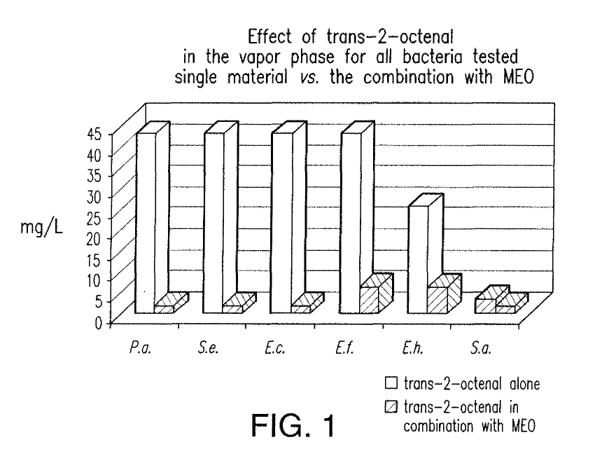

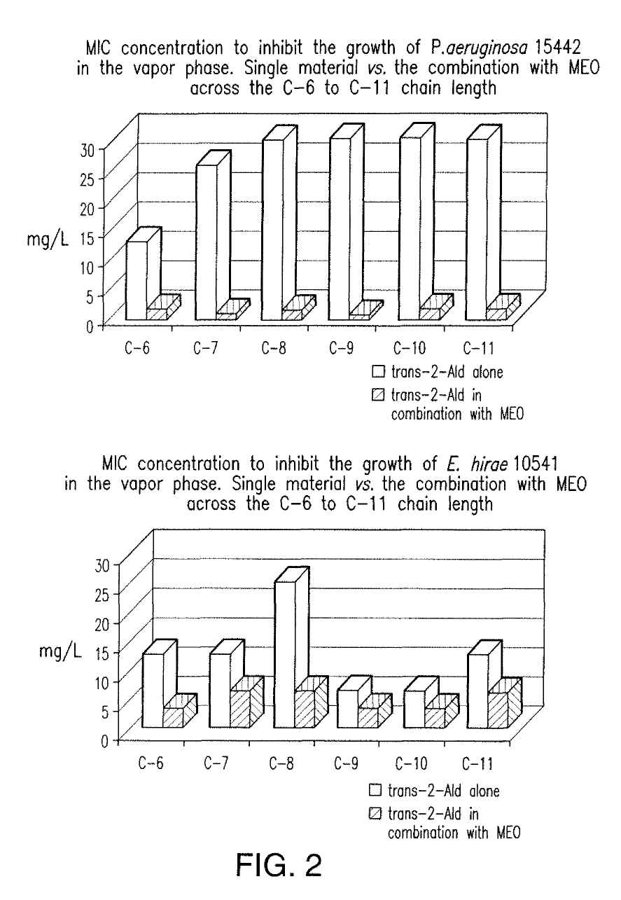

MIC's of trans-2-octenal, alone and in combination with MEO, in the vapor phase against agar plates of Escherichia coli ATCC 10536, Salmonella enterica ATCC 13311, Pseudomonas aeruginosa ATCC 15422, Staphylococcus aureus ATCC 6538, Enterococcus faecalis ATCC 29212, and Enterococcus hirae ATCC 10541 are shown in FIG. 1. The MIC to inhibit the growth of Pseudomonas aeruginosa ATCC 15422 and Enterococcus hirae ATCC 10541 in the vapor phase is shown in FIG. 2.

As shown above, binary combinations of α, β-unsaturated aliphatic aldehydes and MEO showed a synergistic effect in the vapor phase to inhibit the growth of bacteria. Higher chain lengthed aldehydes showed a higher activity against the gram positive bacteria in this example (Staphylococcus aureus ATCC 6538, Enterococcus faecalis ATCC 29212, and Enterococcus hirae ATCC 10541).

MIC values were also determined for trans-2 octenal, octanal formulations, and MEO in solution and in the vapor phase. The results are shown below in Table 6.

TABLE 6
MIC values for trans-2-octenal and octanal formulations in
vapor phase and in solution

| | trans-2- | oetanal | MEO | | | |
|---|---|---|---|---|---|---|
| | octenal in | in | alone | trans-2- | octanal | |
| | vapor | vapor | in vapor | octenal in | in | MEO in |
| | phase | phase | phase | solution | solution | solution |
| | (mg/L) | (mg/L) | (mg/L) | (mg/L) | (mg/L) | (mg/L) |
| P.a. 15442 | 43.0 | 84.8 | 2.0 | >125 | >125 | >62.5 |
| S.e. 13311 | 43.0 | 42.4 | 2.0 | >125 | >125 | >62.5 |
| E.c. 10536 | 43.0 | 21.1 | 2.0 | >125 | >125 | >62.5 |
| E.f. 29212 | 43.0 | 42.4 | 12.8 | >125 | >125 | >62.5 |
| E.h. 10541 | 25.6 | 42.4 | 12.8 | >125 | >125 | >62.5 |
| S.a. 6538 | 3.6 | 7.8 | 2.0 | >125 | >125 | >62.5 |

Table 6 indicates that the tested compositions active in the vapor phase, but not in solution. This demonstrates that antibacterial activity in the vapor phase is distinct from activity in solution. When creating compositions with antibacterial activity, one or more active materials can be selected. Materials active in the vapor phase may not be active in solution (and vice versa). To provide compositions with activity in the vapor phase, consideration need only given to materials that are active in the vapor phase—its' activity in solution is not a consideration. The reverse also holds for compositions active in solution. Therefore active compositions that demonstrate activity in the vapor phase are expected to be quite different from active compositions that demonstrate activity in solution. The differences are not simply due to test methods as both test for antibacterial activity. While not being bound by any particular theory, research suggests that the mechanism of antibacterial activity in the vapor phase and activity in solution is distinct and different from activity in solution. The molecular and/or structural targets on the bacteria can be different.

### Example 2

Seeded brain heart infusion (BHI) agar plates of Escherichia coli ATCC 10536, Salmonella enterica ATCC 13311, Pseudomonas aeruginosa ATCC 15422, Staphylococcus aureus ATCC 6538, Enterococcus faecalis ATCC 29212, and Enterococcus hirae ATCC 10541 were placed in sealed 7 L acrylic boxes and exposed to vapors of the compositions shown below. A control was established in which the agar plates was not treated with vapors.

Composition A: 9.3 mg/L of trans-2-hexenal
Composition B: 9.3 mg/L of trans-2-heptenal
Composition C: 9.3 mg/L of 2,4-heptadienal
Composition D: 7.1 mg/L of trans-2-hexenal
Composition E: 7.1 mg/L of 2,4-heptadienal
Composition F: 7.1 mg/L of 2,4-heptadienal+citronellol (1:1)
Composition G: 9.3 mg/L of citronellal+citral DMA (1:1)
Composition H: 9.3 mg/L of benzaldehyde+citral DMA (1:1)
Composition I: 9.3 mg/L of furfural+citronellol (1:1)
Composition J: 7.1 mg/L of citral+citronellal (1:1)
Composition K: 7.1 mg/L of trans-2-hexenal+citronellol (1:1)
The following results were obtained:

TABLE 7
Vapor Phase Activity Against Bacteria

| | S.a. | E.f. | E.h. | E.c. | S.e. | P.a. |
|---|---|---|---|---|---|---|
| | 6538 | 29212 | 10541 | 10536 | 13311 | 15442 |
| Composition | +++ | +++ | + | +++ | +++ | +++ |
| A | | | | | | |
| Composition | +++ | +++ | +++ | +++ | +++ | + |
| B | | | | | | |
| Composition | +++ | +++ | + | +++ | +++ | +++ |
| C | | | | | | |
| Composition | +++ | +++ | + | +++ | +++ | +++ |
| D | | | | | | |
| Composition | +++ | ++ | + | +++ | +++ | ++ |
| E | | | | | | |
| Composition | +++ | − | − | − | − | − |
| F | | | | | | |
| Composition | − | − | − | − | − | − |
| G | | | | | | |
| Composition | ++ | − | − | − | − | − |
| H | | | | | | |
| Composition | − | − | − | − | − | − |
| I | | | | | | |
| Composition | +++ | − | − | − | − | − |
| J | | | | | | |
| Composition | +++ | − | − | ++ | ++ | − |
| K | | | | | | |

KEY:
−No Activity (growth)
+Slight Activity (some growth, but much less than control)
++Medium Activity (pinprick growth)
+++High Activity (no growth)

### Example 3

A detergent base was obtained based on a commercially-available gel detergent product and used as a positive control. To the positive control was added, in separate trials, a fragrance composition containing 0.2 wt % of trans-2-nonenal, trans-2-decenal, trans-2-undecenal, trans-2-dodecenal, and trans-2-tridecenal aldehydes.

The activity log reduction of these compositions was determined against Enterococcus hirae ATCC 10541 and Staphylococcus aureus ATCC 6538 in solution. Each 1 gram of product was diluted with 116 grams of water, stirred with a stir bar for about 10 minutes. An aliquot was added with bacteria, mixed on a vortex mixer and placed in a 50° C. water bath. After 1 hour, sample was diluted in D/E Neutralization broth (Dey and Engley) and plated onto solid media. Surviving bacteria was counted after 1 day incubation at 37° C. The results are shown below in Table 8.

TABLE 8
Activity Log Reduction of Enterococcus hirae ATCC 10541 and
Staphylococcus aureus ATCC 6538 in solution

| | Enterococcus hirae | Staphylococcus aureus |
|---|---|---|
| Composition Tested | ATCC 10541 | ATCC 6538 |
| Detergent base (control) | 1.5 | 3.1 |
| +trans-2-nonenal | 2.2 | 3.9 |
| +trans-2-decenal | 3.6 | >5 |
| +trans-2-undecenal | 4.8 | 4.7 |
| +trans-2-dodecenal | >5 | >5 |
| +trans-2-tridecenal | >5 | 3.9 |

The activity log reduction of a fragrance composition containing trans-2-dodecenal was tested when used in increasing amounts from 0.3% to 0.5%. The composition was tested against Pseudomonas aeruginosa ATCC 15422, Escherichia coli ATCC 10536, Staphylococcus aureus ATCC 6538, and Enterococcus hirae ATCC 10541. The results are shown below in Table 9, in which the anti-microbial composition decreased bacterial activity in a dose-related manner, while providing a hedonically appealing fragrance.

TABLE 9
Activity log reduction of a fragrance
composition containing trans-2-dodecenal

| | | Escherichia | | |
|---|---|---|---|---|
| | Pseudomonas | coli | Staphylococcus | Enterococcus |
| | aeruginosa | ATCC | aureus ATCC | hirae ATCC |
| | ATCC 15422 | 10536 | 6538 | 10541 |
| Detergent | >5 | >5 | 2 | 1 |
| base | | | | |
| (control) | | | | |
| Base + 0.3% | >5 | >5 | 4 | 3 |
| Table 8 | | | | |
| trans-2- | | | | |
| dodecenal | | | | |
| composition | | | | |
| Base + 0.4% | >5 | >5 | >5 | 3.5 |
| Table 8 | | | | |
| trans-2- | | | | |
| dodecenal | | | | |
| composition | | | | |
| Base + 0.5% | >5 | >5 | >5 | >5 |
| Table 8 | | | | |
| trans-2- | | | | |
| dodecenal | | | | |
| composition | | | | |

### Example 4

The following composition was prepared by mixing the following components, in which the percentages are weight percent.

TABLE 10
Antibacterial Aldehyde-Containing
Composition with Fragrance Components

| Component | Amount (wt %) |
|---|---|
| decanal | 36% |
| octanal | 2% |
| nonanal | 2% |
| citronellyl acetate (available from Takasago | 13% |
| Int'l Corp.) | |
| citronellyl nitrile (available from Takasago | 8% |
| Int'l Corp.) | |
| dh citronellal (@10%) (available from | 3% |
| Takasago Int'l Corp.) | |
| ethyl butyrate | 1% |
| hindinol (available from Takasago Int'l Corp.) | 1% |
| iso propoxy ethyl salicylate (available from | 15% |
| Takasago Int'l Corp.) | |
| linalyl acetate synthetic | 9% |
| terpinyl acetate | 10% |
| 3,5,5-trimethylhexyl acetate | 1% |

This composition was shown to have antibacterial activity against all of the bacteria tested, including gram-negative and gram-positive bacteria.

The present invention is not to be limited in scope by the specific embodiments described herein. Various modifications of the invention in addition to those described herein will become apparent to those skilled in the art from the foregoing description and the accompanying figures. Such modifications are intended to fall within the scope of the appended claims.

Patents, patent applications, publications, product descriptions, and protocols are cited throughout this application, the disclosures of each of which is incorporated herein by reference in its entirety for all purposes.

What is claimed is:

1. An antimicrobial composition comprising from about 2 wt % to about 80 wt % of an unsaturated or saturated C₆-C₁₃ straight-chained or branched aliphatic aldehyde.

2. The antimicrobial composition of claim 1, wherein the composition contains from about 4 wt % to about 50 wt % of the unsaturated or saturated C₆-C₁₃ straight-chained or branched aliphatic aldehyde.

3. A product comprising the antimicrobial composition of claim 1.

4. The product of claim 3, wherein the product is selected from the group consisting of bathroom cleansers, kitchen cleansers, deodorizing products, and fragrance products.

5. The product of claim 3, wherein the product is selected from the group consisting of soap, detergent, dishwasher deodorizer, and air sanitizer.

6. A method of preventing malodor in a confined air space, wherein the method comprises introducing the antimicrobial composition of claim 1 to a confined air space.

7. The method of claim 6, wherein the confined space is selected from the group consisting of dishwashers, refrigerators, garbage pails, sink garbage disposals, toilet bowls, laundry hampers, diaper pails, closets, show boxes, storage boxes, cat litter, pet litter boxes, pet cages, pet bedding, gym lockers, gym bags, sneakers, and shoes.

8. The method of claim 6, wherein the antimicrobial composition is active in a vapor phase.
